(19) United States
(12) Patent Application Publication
(10) Pub. No.: US 2006/0003404 A1
(43) Pub. Date: Jan. 5, 2006

(54) Method for specific integration of t7 rna polymerase gene in the chromosome of corynebacterial and the resultant corynebacteria-t7 promoter based shuttle vector system

(76) Inventors: Jahar Kanti Deb, Hauz Khas (IN); Preeti Srivastava, Hauz Khas (IN); Karan Goutam, Cleveland, OH (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/503,790

(22) PCT Filed: Feb. 5, 2003
(86) PCT No.: PCT/IN03/00019

§ 371 Date: Jun. 10, 2005

(30) Foreign Application Priority Data
Feb. 5, 2002 (IN) 96/DEL/2002

(51) Int. Cl. C12P 21/06; C12N 15/74; C12N 1/21

(52) U.S. Cl. 435/69.1; 435/488; 435/252.33

(57) ABSTRACT

The present invention relates to method for obtaining optimum expressed proteins in a transformed gram positive bacteria by specific integration of T7 RNA polymerase gene into the chromosome of a gram positive bacteria exhibiting resistance to aminoglycosides, said method comprising:- digesting an E. coli plasmid with a restriction enzyme- digesting the genomic DNA of said gram positive bacteria- ligating the said digested plasmid to the digested genomic DNA of said gram positive bacteria- transforming the said gram positive bacteria protoplasts with the said ligation mixture of step 2 to yield transformed gram positive bacteria (transformants),- screening the said transformants for kanamycin resistance and aminoglycoside sensitivity to ensure that the targetting of the said plasmid vector into the chromosome of the said gram positive bacteria is successful- cloning of the desired gene in the said vector—culturing the transformant in a suitable culture medium- isolating the expressed proteins from the culture medium

### FIELD OF THE INVETION

This invention relates to a method for specific integration of T7 RNA Polymerase gene into the Chromosome of Corynebacteria and resultant novel Corynebacteria-T7 promoter based shuttle vector and shuttle vector system.

### BACKGROUND OF INVENTION

E. coli has been the host of choice for the production of recombinant proteins since the last many years. However being a gram negative bacteria, the secretion of proteins is limited in E. coli. It forms inclusion bodies from which the recombinant protein has to be isolated by using harsh chemical treatment. Besides, not only is the reducing environment in the cytoplasm not conducive for several proteins, the periplasmic space of this bacteria has got several proteases, which degrade the expressed proteins reducing their yield considerably.

For these reasons, the use of naturally secreting organisms for protein production may be highly advantageous. An alternative to the E. coli system would be the gram-positive bacteria such as Bacillus subtilis which is very active in secreting the protein into the medium. But, it suffers from a major drawback. It also secretes large amount of proteases, which degrade the recombinant protein. Besides, it forms spores, which require harsh and special condition for killing and can spread easily. Non-pathogenic gram positive soil corynebacteria are attractive alternative systems for gene expression. They are non-sporulating and have very little proteolytic activity. E. Coli is not a GRAS organism.

Compared with other established high G+C gram-positive hosts such as certain Streptomyces species, corynebacteria has got some advantages; there are no complex cellular differentiation steps such as mycelium formation or sporulation during growth. Also corynebacteria do not have the disadvantage of severe genetic instabilities, as are known to occur in Streptomyces species (Liebl et al. , 1992). One major advantage of these bacteria over low G+C gram-positive hosts such as Bacillus subtilis or Staphylococcus species is the rather broad acceptance of heterologous signals including gram-negative promoters (Morinaga et al. , 1987). They are rapid growers (ₘₐₓ˜0. 35 h⁻¹ on synthetic medium) and do not form clumps (Eggeling and Sahm, 2001). The mycolic acids in Corynebacterium have a comparatively simple structure. Their total carbon number in different Corynebacterium species is in the range of 22-38, whereas it is 34-48 in Rhodococcus, 48-66 in Gordonia, and 64-78 in Tsukamurella (Eggeling and Sahm, 2001). The most complex mycolic acids are present in Mycobacteria where the total carbons can go up to 90. Besides, soil corynebacteria have very little proteolytic activity and they come under the category of GRAS (Generally Regarded As Safe) organisms. Corynebacteria represent a model organism suitable for functional studies of expression of mycobacterial genes (Puech et al. , 2001). The heterologous expression of mycobacterial antigens in C. glutamicum has proved to be effective (Salim et al. , 1997; Puech et al. , 2000).

Food grade corynebacteria are used industrially for the production of several amino acids. Currently, more than one million tons of amino acids are produced annually with bacteria. The major amino acids are glutamate, lysine, phenylalanine, glutamine, arginine, tryptophan, threonine, isoleucine and histidine. A total of 700,000 tons of glutamate alone are produced annually and 300,000 tons of lysine. The quantity of isoleucine produced is about 400 tons. The amino acids have many different applications. They are used as flavoring agent (glutamate), as a feed additive (lysine, threonine), as a building block in chemical synthesis (phenylalanine, valine) or for pharmaceutical purposes (histidine). Isoleucine, leucine and valine are constituents of infusions and special dietary products (Eggeling et al, 1997). Because of the high profile of these organisms in health and industry, the genetics of corynebacteria have been fairly well researched (Jetten and Sinskey, 1995).

A number of expression vectors of E. coli have been developed using strong promoters, the foremost of which has been the T7 promoter of bacteriophage T7. T7 based expression systems are widely used for large scale over expression of recombinant proteins in both bacterial and eukaryotic cells. The system makes use of the T7 RNA polymerase which is a highly active enzyme elongating RNA chains eight times faster than E. coli RNA polymerase.

The expression from the T7 promoter is under the control of RNA polymerase of the same bacteriophage called T7 RNA polymerase. This is very specific for T7 promoter. Plasmid pGP1-2 has the RNA polymerase gene of bacteriophage T7 under the control of P_{L} promoter inducible by thermosensitive c1 repressor of bacteriophage λ and a kanamycin resistance gene marker (Tabor,S and Richardson, C. C. , 1985). This is T7 expression system in E. coli.

One such experiments in which the T7 RNA polymerase has been integrated in the chromosome of E. coli and subsequently the gene of the protein to be expressed is inserted in a T7 promoter based expression vector system is known in the art.

While there are several such systems in E. coli, no such vector system is known in Corynebacteria the advantages of which over E. coli have been discussed hereinbefore. Also most promoter systems in Corynebacteria are known to produce poor expression yields.

The main object of this invention is to demonstrate a method for effectuating a specific integration of T7 RNA polymerase gene into the chromosomal DNA of Corynebacteria in order to obtain high success rates of getting the desired strain.

Another object of this invention is to eliminate the cost and time intensive process of screening of random samples.

Further, an object of this invention is to construct an E. coli Corynebacteria shuttle vector (pBKET29aS) T7).

Yet another object is to construct Corynebacteria—T7 based shuttle vector system.

### SUMMARY OF THE INVENTION

Accordingly, the said invention involves a method of integration of T7 RNA polymerase gene into the chromosome of corynebacteria (C. acetoacidophilum). The said method for specific integration uses an E. coli plasmid pGP1-2 carrying the gene for kanamycin resistance, the genes for T7 RNA Polymerase and the cI repressor. This plasmid is digested with restriction enzyme Bam HI and ligated to the Sau3AI digested genomic DNA of C. acetoacidophilum. Thereafter a ligation mixture is used to transform C. acetoacidophilum protoplasts. Transformants are then screened for Kanamycin resistance and senitivity to aminoglycoside such as streptomycin, to ensure targeting of plasmid vector pGP1-2 into the chromosome of C. acetoacidophilum. The process will lead to the modification of the chromosomal DNA of C. acetoacidophilum, which now enables the said bacterium designated B-30T7R to express T7 RNA polymerase. These cells are then processed for isolation of pGP 1-2 to detect plasmid DNA. The absence of plasmid DNA establishes that the plasmid has integrated into the chromosome of the cell. It was also noticed that this chromosomal integration did not affect the growth of this strain.

The instant invention has a number of advantages over the existing prior art. Here, the screening of the clones is simple and cost effective. Unlike other systems, no expensive chemical is required for regulation. Besides, no adverse effect is noticed on cell growth since the chromosomal integration occurs at nonessential gene (i. e. nonessential for the basic cell metabolism). Such an integration of T7 RNA polymerase gene at the non-essential portion of the chromosome ensures the stable maintenance of the culture. Another advantage is that there is no protease induction when the temperature is raised to 40° C. Regulation of protein expression is by means of a thermosensitive cI repressor, which further cuts down costs by doing away with expensive chemicals.

Accordingly, the instant invention provides a method for obtaining optimum expressed proteins in a transformed gram positive bacteria by specific integration of T7 RNA polymerase gene into the chromosome of a gram positive bacteria exhibiting resistance to aminoglycosides, said method comprising:
digesting an E. coli plasmid with a restriction enzyme
digesting the genomic DNA of said gram positive bacteria
ligating the said digested plasmid to the digested genomic DNA of said gram positive bacteria
transforming the said gram positive bacterial protoplasts with the said ligation mixture of step 2 to yield transformed gram positive bacteria (transformants),
screening the said transformants for kanamycin resistance and aminoglycoside sensitivity to ensure that the targetting of the said plasmid vector into the chromosome of the said gram positive bacteria is successful
cloning of the desired gene in the said vector
culturing the transformant in a suitable culture medium
isolating the expressed proteins from the culture medium

The instant invention also provides for a novel shuttle vector pBKET29aS comprising:
a corynebacterial plasmid pBK2 derived from the cryptic plasmid pBL1 of B. lactofermentum
a novel origin of replication p15A obtained from E. coli plasmid pACYC184
a novel spectinomycin resistance gene cassette Spc^{r} obtained from E. coli plasmid pDG1726,
a T7 promoter cassette obtained from E. coli plasmid pET29a prepared by conventional means

Further, the instant invention provides for a novel shuttle vector system comprising
said novel shuttle vector and
gram negative bacteria or gram positive bacteria having a T7 RNA polymerase gene integrated into the bacterial chromosome,
   wherein said novel shuttle vector is placed in the said bacteria.

### BRIEF DESCRIPTION OF THE DRAWINGS

FIG. 1 outlines the strategy adopted for the integration of T7 RNA polymerase gene of plasmid pGP1-2 into the genomic DNA of C. acetoacidophilum.

FIG. 2 shows the construction of shuttle vector pBK2s by the ligation of partially digested and gel eluted fragment of pBK2 with similarly treated fragment of pACYC184.

FIG. 3 shows the steps involved in the construction of vector pBK2Spcs derived from pBK2s

FIG. 4 depicts the detailed map of vector pET29a as well as the gene sequence showing the multiple cloning site and T7 terminator

FIG. 5 shows the steps involved in the construction of vector pBKET29aS

FIG. 6 depicts a detailed map of plasmid pBKET29aS

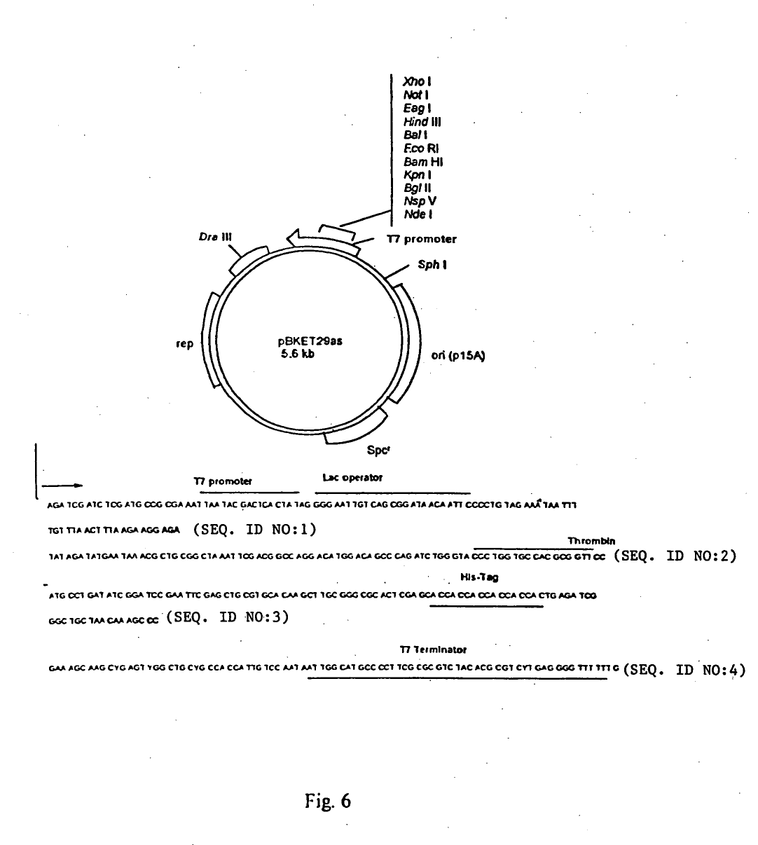

FIG. 7 shows the construction of vector pBKET29aSGAL

FIG. 8 shows the construction of vector pBKET29aSsk

### DETAILED DESCRIPTION WITH RESPECT TO THE ACCOMPANYING DRAWINGS

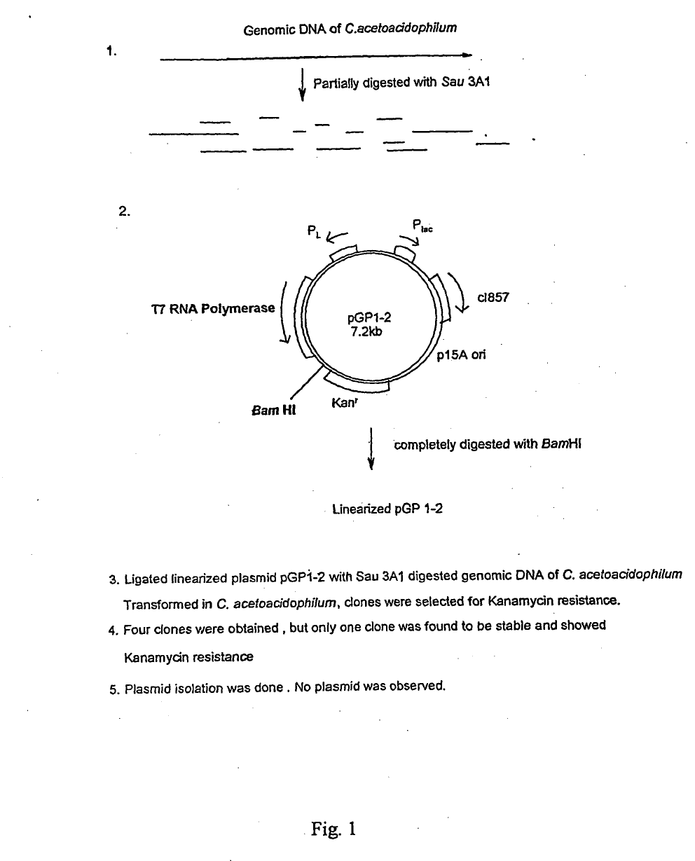

DNA of Corynebacteria (Corynebacterium acetoacidophilum) comprising a gene of T7 RNA polymerease has been produced wherein the said gene of T7 RNA polymerase has been integerated into the said DNA of the said Corynebacteria at the site of the streptomycin resistant gene (FIG. 1). The streptomycin resistant gene of C. acetoacidophilum has been chosen as the site of integration of T7 RNA Polymerase gene for two reasons.

Firstly, the gene is nonessential for the growth of the organism and therefore integration at this site does not impede the growth of the transformed bacterial culture. Secondly the screening of T7 RNA polymerase integrated transformants is easy.

The resistance of the corynebacteria to streptomycin is very high, the streptomycin concentration being as high 10 mg/ml.

The said recombinant DNA has been placed under the control of thermoinducible P_{L} promoter which has been integrated into the chromosome of Corynebacterium acetoacidophilum. The recombinant proteins that can be include biopharmaceutical proteins such as Interleukin 2, GCSF, GMCSF, γ-Interferon, Human growth hormone and enzymes such as Xylanases, Cellulases, Lipases etc.

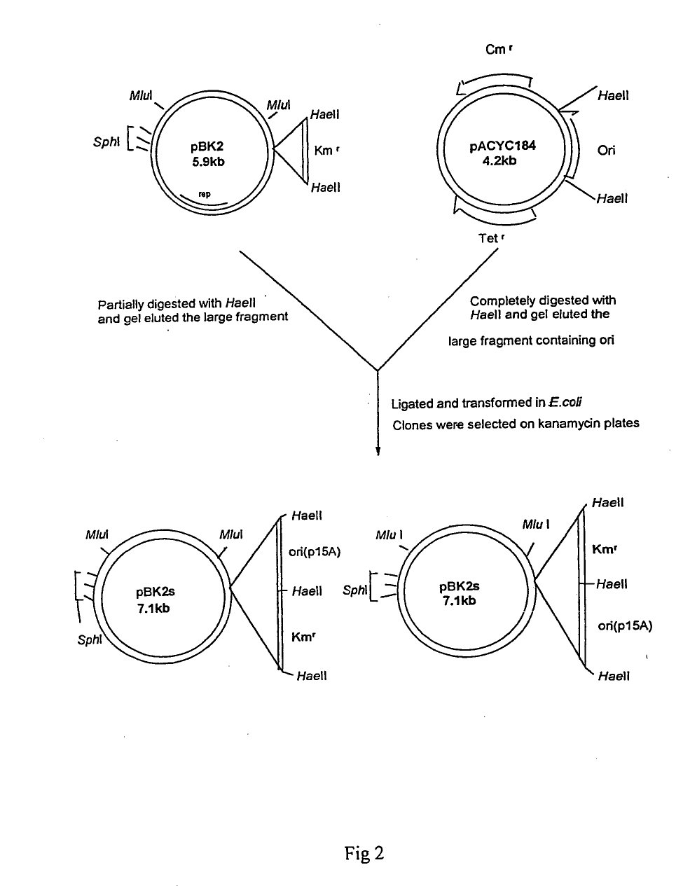

The E. coli-C. acetoacidophilum shuttle vector is constructed by using a series of new vectors which were developed by using corynebacterial plasmid pBK2 as the starting vector. Since the resistance marker in this plasmid and that in C. acetoacidophilum containing T7 RNA polymerase gene integrated into its genome is kanamycin, a different marker has to be inserted in the plasmid containing T7 promoter cassette. For this purpose, initially a recombinant E. coli-C. acetoacidophilum shuttle vector, pBK2s, was developed by inserting the ori of pACYC184 plasmid as shown in FIG. 2.

E. coli plasmid pACYC184 was completely digested with HaeII. This plasmid has eleven sites of HaeII. The larger fragment containing the p15A origin was gel eluted. Corynebacterial plasmid pBK2 was partially digested with HaeII, which has two sites in the plasmid. The two digests were ligated and used to transform E. coli (DH5a) competent cells. Clones were selected for kanamycin resistance. Plasmid isolated both on small and large scale from these clones showed the presence of the desired 7. 1 kb plasmid (pBK2s).

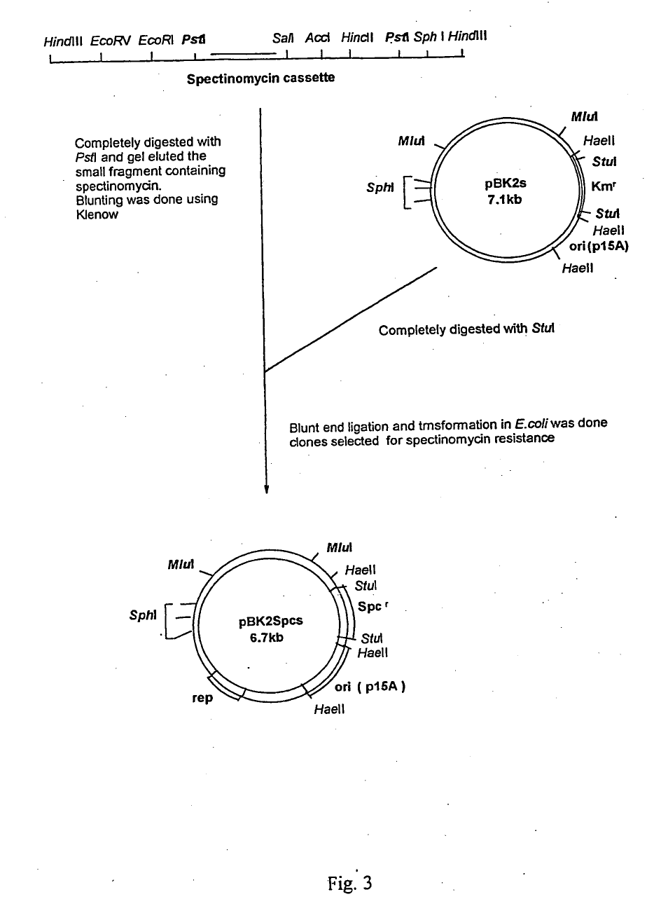

The kanamycin resistance gene of plasmid pBK2s was replaced by spectinomycin resistance gene by inserting spectinomycin resistance cassette of E. coli plasmid pDG1726 to form pBK2SpcS which is FIG. 3. Plasmid pDG1726 was digested with PstI which has two sites in the plasmid. The ends were filled by with Klenow polymerase. The smaller fragment containing the spectinomycin resistance gene cassette was gel eluted. Plasmid pBK2s was digested with StuI. Thereafter the two digests were ligated to form the plasmid pBK2Spcs.

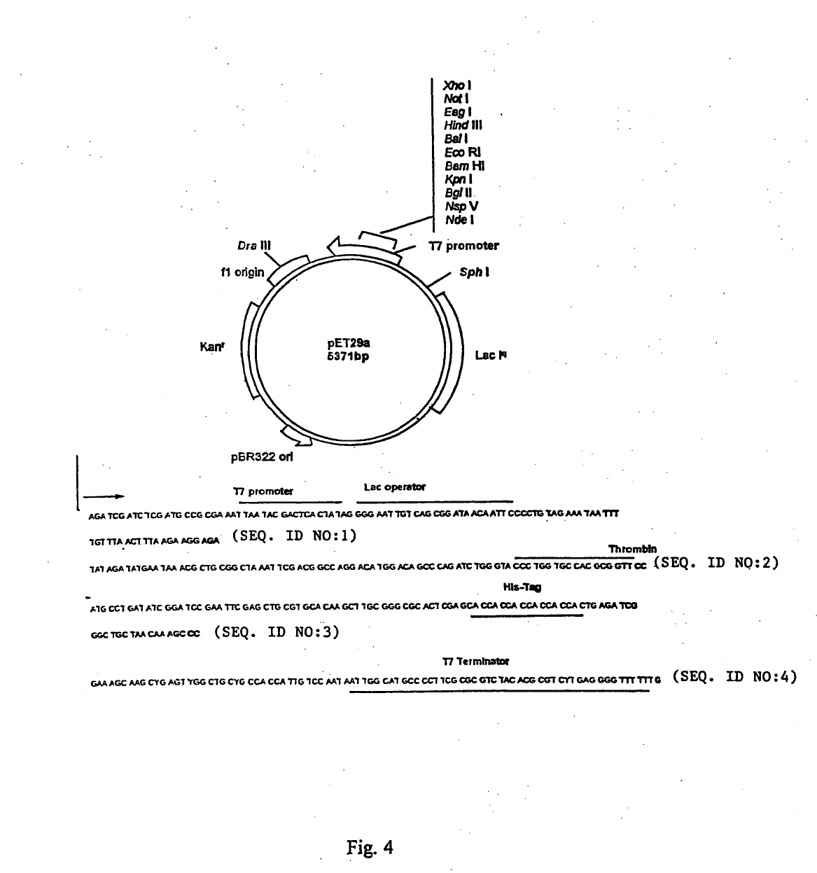

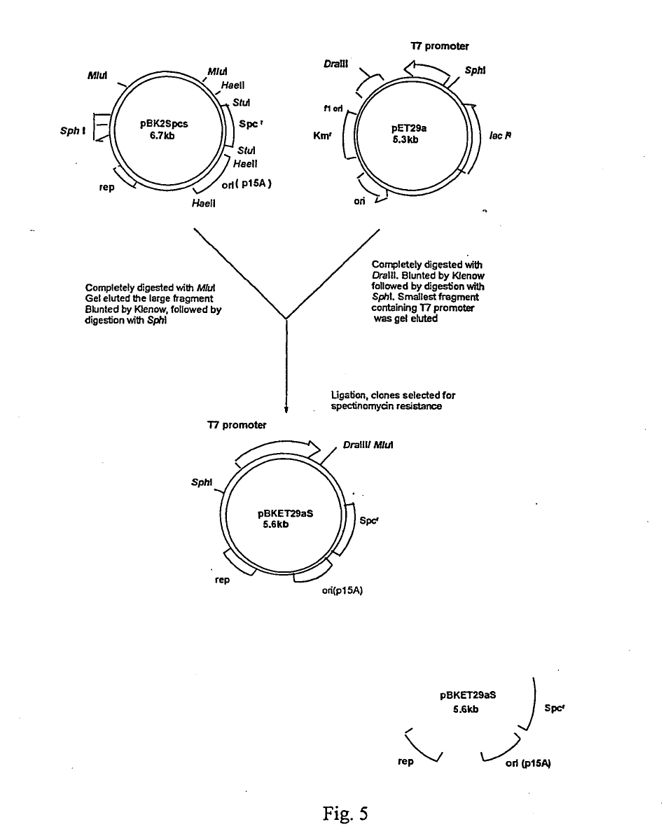

A T7 promoter cassette was introduced into the resulting pBK2Spcs from the plasmid pET29a (FIG. 4) according to the scheme shown in FIG. 5 to develop pBKET29as. E. coli plasmid pET29a was digested with DraIII, ends were filled with Klenow polymerase. This was followed by digestion with SphI. The smallest fragment containing T7 promoter cassette was gel eluted. Plasmid pBK2Spcs was digested with MluI, ends filled with Klenow polymerase, followed by digestion with SphI. The two fragments were ligated and clones were selected for spectinomycin resistance.

The said new vector is a shuttle vector exhibiting the ability to replicate in two different hosts, E. coli and C. acetoacidophilum.

The said shuttle vector contains T7 promoter, transcriptional terminators, ribosome binding site (RBS), multiple cloning site, spectinomycin resistance gene marker p15A and pBL1 origin of replication.

The said shuttle vector was introduced in the corynebacterial strain containing the modified DNA for the expression of T7 RNA polymerase. The T7 RNA polymerase produced by the modified DNA binds to the T7 promoter of the vector and thereby transcribes the DNA. The expression is regulatable by a thermosensitive cI repressor.

A shift in temperature difference from 30° C. to 40° C. leads to the expression of the foreign gene

This invention demonstrates for the first time the efficient functioning of T7 RNA polymerase and T7 promoter function in corynebacteria. The inventions also demonstrates an effective and regulatable gene expression system (Corynebacteria - pBKET29aS) in Corynebacteria.

The said corynebacteria-pBKET29aS expression vector system of the present invention is designed more particularly for the production of products of commercial importance. These may be any proteins whose gene when cloned in the right frame will lead to the corresponding product.

The strain of the said expression vector system is nonpathogenic, GRAS grade gram-positive organism, which is low in proteolytic activity and does not produce any toxins. The said strain of the said system provides high level expression of foreign genes. No expensive chemicals are required for the induction of gene expression.

T7 promoter is a very strong promoter and this invention demonstrates for the first time the efficient expression of this promoter in corynebacteria. The T7 RNA polymerase is able to make transcripts of almost any DNA that is placed under the control of a T7 promoter. T7 RNA polymerase is known to elongate chains about eight times faster than does E. coli RNA polymerase. In Corynebacteria, the level of expression achieved is higher using this system than it is in E. coli using similar system. Major amount of the protein-was secreted in the medium unlike in E. coli where the protein mostly remained within the cytoplasm of the cell. Secretion in the medium means that fewer proteins will be present as contaminants and therefore is attractive from the down stream point of view which is the most expensive stage of an industrial process.

The DNA of the shuttle vector system (pBKET29aS) has been produced by combining the fragments of corynebacterial plasmid pBL1, E. coli plasmid pACYC184, E. coli plasmid pDG1726 and a T7 promoter cassette, which is obtained from the plasmid pET29a of E. coli.

The combination of the said DNA of corynebacteria and the said DNA of the shuttle vector directs the T7 promoter which is specific for said T7 RNA polymerase to transcribe the polypeptide or proteins selectively after temperature induction as a result of expression of said polymerase in a highly efficient manner in corynebacteria.

The invention shall now be described with the help of some examples:

### EXAMPLE I

### Expression of Streptokinase

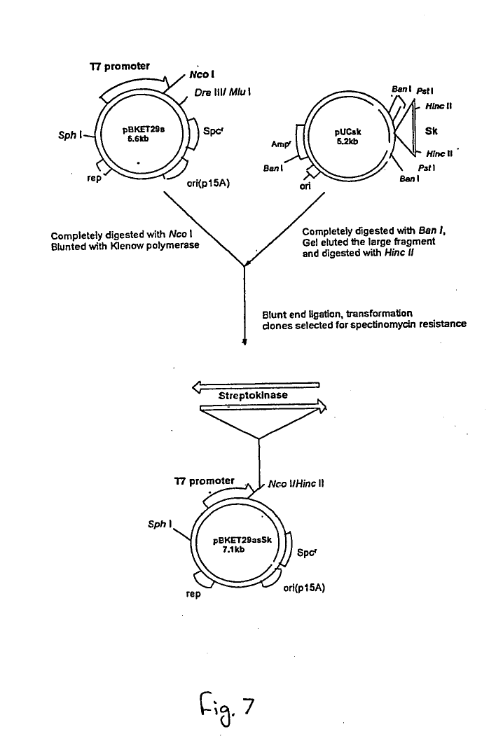

Streptokinase has shown itself to be an efficacious agent in the clinical treatment of acute myocardial infarction following coronary thrombosis and has served as a thrombolytic agent for almost three decades. Streptokinase activates plasminogen to plasmin by forming an activator complex with plasminogen. The SK plasminogen complex forms the basis of the active-center modified thrombolytic prodrug anistreplase. Streptokinase gene was obtained from the plasmid pUCsk. Plasmid pUCsk contains 2. 5 kb PstI fragment carrying streptokinase gene in pUC19. This plasmid was digested with BanI and the larger fragment was gel eluted. This fragment was further digested with HincII. Plasmid pBKET29aS was digested with NcoI and the ends were filled with Klenow polymerase. The two fragments were ligated and transformed in the B-30T7R protoplasts. Clones were selected for spectinomycin resistance. The positive clones carrying pBKET29asSk (FIG. 7) were identified by the assay based on clear zones on plasminogen-milk agar plates.

Family of proteins that can be expressed in the corynebacterial host by the novel shuttle vector system include Biopharmaceutical proteins such as Interleukin 2, GCSF, GMCSF, γ-Interferon, Human growth hormone and Enzymes such as Xylanases, Cellulases, Lipases etc.

### Comparison of T7 and tac promoter (Expression of Streptokinase)

The intracellular and extracellular expression of Streptokinase in T7 promoter based shuttle vector system (Table 2) vis a vis expression driven by tac promoter is tabulated below (Table 1).

While both are C. acetoacidophilum based promoter systems, the tables show that the expression of both intracellular and extracellular protein is considerably higher in T7 based promoter system as compared to tac promoter system (Table. 3).

TABLE 1
Intracellular and Extracellular Activities of GST-Streptokinase
fusion driven by tac promoter

| | Intracellular | | Extracellular | | Total | |
|---|---|---|---|---|---|---|
| | U · ml⁻¹ · | | U · ml⁻¹ | | Activity | Extracellular/ |
| Sample | Total U | | Total U | | (U) | Intracellular |
| C. aceto- | 36 | 1800 | 560 | 28,000 | 29,800 | 15.6 |
| acidophilum | | | | | | |
| pBKGEXm2sk | | | | | | |

TABLE 2
Intracellular and Extracellular Activities
of Streptokinase driven by T7 promoter

| | | | | | | Extra- |
|---|---|---|---|---|---|---|
| | Intracellular | | Extracellular | | Total | cellular/ |
| | U · ml⁻¹ | | U · ml⁻¹ | | Activity | Intra- |
| Sample | Total U | | Total U | | (U) | cellular |
| C. aceto- | 1600 | 1600 | 2,000 | 1,00,000 | 1,01,600 | 62.5 |
| acidophilum | | | | | | |
| (B-30T7R) | | | | | | |
| pBKET29ask | | | | | | |

TABLE 3
Comparison of Streptokinase expression
in E. coli and corynebacteria driven
by T7 promoter

| | Intracellular | | Extracellular | | Total |
|---|---|---|---|---|---|
| | U · ml⁻¹ | | U · ml⁻¹ | | Activity |
| Sample | Total U | | Total U | | (U) |
| E. coli(BL21) | 1200 | 1200 | 1270 | 63000 | 64200 |
| pBKET29aSsk | | | | | |
| E. coli(DH5α) | 1800 | 1800 | 1450 | 72500 | 74,300 |
| pGP1-2 | | | | | |
| pBKET29aSsk | | | | | |
| C. aceto- | 1600 | 1600 | 2,000 | 1,00,000 | 1,01,600 |
| acidophilum | | | | | |
| (B-30T7R) | | | | | |
| pBKET29aSsk | | | | | |

### EXAMPLE II

### Expression of β-galactosidase (beta-galactosidase)

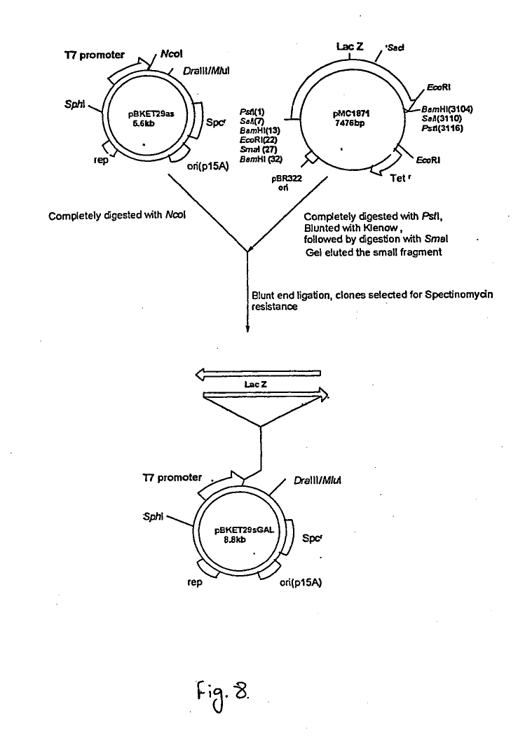

The enzyme catalyzes the hydrolysis of lactose and many beta-D-galactopyranosides. The DNA sequence of the gene (LacZ) has been determined and it encodes a 116,000 dalton polypeptide. β-galactosidase is often used as a reporter gene. An E. coli plasmid pMC1871 was used for this purpose. This plasmid was digested with PstI followed by SmaI. The larger fragment containing the gene for β-galactosidase was gel eluted. This was ligated to the CIAP treated NcoI digested pBKET29aS vector. Clones were selected for spectinomycin resistance. Positive clones were screened on X-gal and IPTG plates. The vector so constructed was designated pBKET29aSGAL (FIG. 8)

### Protease Assay

After heat induction, the cells were sonicated and centrifuged. Fixed amount of Bovine serum albumin was incubated with a definite quantity of the culture supernatant at 37° C. for various time intervals and the protein analysed by SDS-PAGE to monitor proteolytic degradation.

The subject application is a mere statement of invention and should not in anyway be construed upon as to restrict the scope of the invention. The account given herein is in no way exhaustive of the details of the invention and should therefore be dealt with accordingly.

### REFERENCES

1. Deb, J. K. , Malik, S. , Ghosh, V. K. , Mathai, S. and Sethi, R. (1990) Intergenic protoplast fusion between xylanase producing Bacillus subtilis LYT and C. acetoacidophilum ATCC 21476. FEMS Microbiol. Lett. 71, 287-292.
2. Eggeling, L. and Sahm, H. (2001). The cell wall barrier of Corynebacterium glutamicum and amino acid efflux. J. Biosci. Bioengg. 92, 201-213.
3. Eggeling, L. , Morbach, S. and Sahm, H. (1997). The fruits of molecular physiology: engineering the L-isoleucine biosynthesis pathway in Corynebacterium glutamicum. J. Biotechnol. 56, 167-182.
4. Jetten, Mike S. M. and Sinskey, A. J. (1995). Recent Advances in the Physiology and Genetics of Amino acid-producing bacteria. Crit. Rev. Biotech. , 15, 73-103.
5. Klessen, C. , Schmidt, H. , Ferreti, J. , and Malke, H (1988) Tripartite streptokinase gene fusion vectors for gram-positive and gram-negative procaryotes. Mol. Gen. Genet. , 212, 295-300.
6. Labarre, J. , Reyes, O. , Guyonvarch, A. and Leblon, G. (1993) Gene-replacement, integration and amplification at the gdhA locus of Corynebacterium glutamicum. J. Bacteriology 175:1001-1007.
7. Liebl, W. , Sinskey, A. J. and Schleifer, K. H. (1992). Expression, secretion and processing of staphylococcal nuclease by Corynebacterium glutamicum. J. Bacteriol. 174, 1854-1861.
8. Morinaga, Y. , Tsuchiya, M. , Miwa, K. , Sano, K. (1987) Expression of E. coli promoters in Brevibacterium lactofermentum using the shuttle vector pEB003. J. Biotechnol. 5, 305-312.
9. Puech, V. , Bayan, N. , Salim, K. , Leblon, G. and Daffe, M. (2000). Characterization of the in vivo acceptors of the mycolyl residues transferred by the corynebacterial PSI and the related mycobacterial antigens 85. Mol. Microbiol. , 35, 1026-1041
10. Puech, V. , Chami, M. , Lemassu, A. , Laneelle, M. A. , Schiffier, B. , Gounon, P. , Bayan, N. , Benz, R. and Daffe, M. (2001). Structure of the cell envelope of corynebacteria: importance of the non-covalently bound lipids in the formation of the cell wall permeability barrier and fracture plane. Microbiology 147, 1365-1382.
11. Reyes, O. , Guyonvarch, A. , Bonamy, C. , Salti, V. , David, F. and Leblon, G. (l991). Integron-bearing vectors: a method suitable for stable chromosomal integration in highly restrictive Corynebacteria. Gene 107:61-68.
12. Salim, K. , Haedens, V. , Content, J. , Leblon, G. and Huygen, K. (1997) Heterologous expression of the M. tuberculosis gene encoding antigen 85A in C. glutamicum. Appl. Environ. Microbiol. , 63, 4392-4400.
13. Studier, F. , and Moffat, B. (1986) Use of Bacteriophage T7 RNA Polymerase to Direct Selective High-level Expression of Cloned Genes, J. Mol. Biol. , 189, 113
14. Tabor, S. and Richardson, C. (1985) A bacteriophage T7 RNA polymerase/promoter system for controlled exclusive expression of specific genes. Proc Natl Acad Sci USA 82:1074-1078.

We claim:

1. A method for obtaining optimum expressed proteins in a transformed gram positive bacteria by specific integration of T7 RNA polymerase gene into the chromosome of a gram positive bacteria exhibiting resistance to aminoglycosides, said method comprising:
digesting an E. coli plasmid with a restriction enzyme;
digesting the genomic DNA of said gram positive bacteria;
ligating the said digested plasmid to the digested genomic DNA of said gram positive bacteria;
transforming said gram positive bacterial protoplasts with
said ligation Mixture of step 2 to yield transformed gram positive bacteria (transformants);
screening said transformants for kanamycin resistance and aminoglycoside sensitivity to ensure that the targeting of said plasmid vector into the chromosome of said gram positive bacteria is successful;
cloning of the desired gene in said vector;
culturing the transformant in a suitable culture medium; and
isolating the expressed proteins from the culture medium.

2. A method as claimed in claim 1 wherein said gram positive bacteria is Corynebacterium acetoacidophilum.

3. A method as claimed in claim 1 wherein said E. coli plasmid is pGP1-2 carrying the genes for kanamycin resistance, T7 RNA polymerase and cI repressor.

4. A method as claimed in claim 1 wherein said restriction enzyme for digesting E coli plasmid is Bam HI.

5. A method as claimed in claim 1 wherein said restriction enzyme for digesting genomic DNA of gram-positive bacteria is Sau3A1.

6. A method as claimed in claim 1 wherein said aminoglycoside is streptomycin.

7. A method as claimed in claim 1 wherein the proteins expressed are biopharmaceutical proteins and enzymes.

8. A method as claimed in claim 1 wherein the proteins expressed include at least one selected from the group consisting of Interleukin 2, GCSF, GMSF, γ-lnterferon, and Human Growth Hormone.

9. A method as claimed in claim 1 wherein the proteins expressed include Xylanases, Cellulases and lipases.

10. A shuttle vector pBKET29aS comprising:
a corynebacterial plasmid pBK2 derived from the cryptic Plasmid pBL1 of B. lactofermentum;
a origin of replication p15A obtained from E. coli plasmid pACYC184;
a spectinomycin resistance gene cassette Spc^{r} obtained from E. coli plasmid pDG1726; and
a T7 promoter cassette obtained from E. coli plasmid pET29a.

11. A shuttle vector as claimed in claim 10 which is capable of replicating in at least one of E. coli and Corynebacterium acetoacidophilum.

12. A shuttle vector as claimed in claim 10 wherein the shuttle vector expresses proteins and said expression is regulatable by thermosensitive cI repressor.

13. A shuttle vector as claimed in claim 10 further comprising a thermosensitive cI repressor, wherein the foreign genes are expressed by a shift in temperature.

14. A shuttle vector as claimed in claim 13 wherein said temperature difference may range from 30° C. to 40° C.

15. A process of preparing a shuttle vector pBKET29aS of 10 comprising construction of at least one of plasmid pBK2s, pBK2Spcs and pBKET29aS.

16. A process of preparing a shuttle vector as claimed in claim 15 wherein said plasmid Pbk2s is derived from said plasmid pBK2 of C. acetoacidophilum
by digesting said plasmid pBK2 partially with HaeII,
treating the digested plasmid with alkaline phosphatase, and
ligating the digested plasmid with origin of replication p15A obtained from E. coli plasmid pACY184 after HaeII digestion.

17. A process of preparing a shuttle vector as claimed in claim 15 wherein said plasmid pBK2Spcs is derived from said plasmid pBK2s
by digesting said plasmid pBK2 with StuI,
treating the digested plasmid with alkaline phosphatase, and
ligating the digested plasmid with spectinomycin resistance gene cassette obtained from E. coli plasmid pDG1726 after PstI digestion and blunting with Klenow polymerase.

18. A A process of preparing a shuttle vector as claimed in claim 15 wherein said plasmid pBKET29aS is derived from the said plasmid pET29a and pBK2Spcs by:
Digesting said plasmid pETa with DRAIII,
Blunting the digested plasmid with Klenow polymerase,
Digesting said blunted plasmid with SphI,
Gel eluting the smallest fragment containing T7 promoter cassette,
Digesting said plasmid pBK2Spcs with MluI,
Blunting the digested plasmid with Klenow polymerase,
Digesting said blunted plasmid with SphI,
Gel eluting the largest fragment, and
Ligating the digested fragments obtained from pET29a and pBK2Spcs.

19. A shuttle vector system comprising the shuttle vector of claim 10 and gram negative bacteria or gram positive bacteria having a T7 RNA polymerase gene integrated into the bacterial chromosome, wherein said shuttle vector is placed in said bacteria.

20. A shuttle vector system as comprising the shuttle vector of claim 10 and E. coli or gram positive bacteria having a T7 RNA polymerase gene integrated into the bacterial chromosome, wherein said shuttle vector is placed in said bacteria.

21. A shuttle vector system as comprising the shuttle vector of claim 10 and C. acetoacidophilum or gram positive bacteria having a T7 RNA polymerase gene integrated into the bacterial chromosome, wherein said shuttle vector is placed in said bacteria.

22. The shuttle vector of claim 10 further comprising a thermosensitive cI repressor.

23. A process of preparing the shuttle vector pBKET29aS of claim 11 comprising construction of at least one of plasmid pBK2s, pBK2Spcs and pBKET29aS.

24. A process of preparing the shuttle vector pBKET29aS of claim 12 comprising construction of at least one of plasmid pBK2s, pBK2Spcs and pBKET29aS.

25. A process of preparing a shuttle vector pBKET29aS of claim 13 comprising construction of at least one of plasmid pBK2s, pBK2Spcs and pBKET29aS.

26. A process of preparing a shuttle vector pBKET29aS of claim 14 comprising construction of at least one of plasmid pBK2s, pBK2Spcs and pBKET29aS.

27. A process of preparing a shuttle vector of claim 23 wherein said plasmid Pbk2s is derived from the said plasmid pBK2 of C. acetoacidophilum by
digesting said plasmid pBK2 partially with HaeII,
treating the digested plasmid with alkaline phosphatase, and
ligating the digested plasmid with origin of replication p15A obtained from E. coli plasmid pACY184 after HaeII digestion.

28. A process of preparing a shuttle vector of claim 24 wherein said plasmid Pbk2s is derived from the said plasmid pBK2 of C. acetoacidophilum by
digesting said plasmid pBK2 partially with HaeII,
treating the digested plasmid with alkaline phosphatase, and
ligating the digested plasmid with origin of replication p15A obtained from E. coli plasmid pACY184 after HaeII digestion.

29. A process of preparing a shuttle vector of claim 25 wherein said plasmid Pbk2s is derived from the said plasmid pBK2 of C. acetoacidophilum by
digesting said plasmid pBK2 partially with HaeII,
treating the digested plasmid with alkaline phosphatase, and
ligating the digested plasmid with origin of replication pl5A obtained from E. coli plasmid pACY184 after HaeII digestion.

30. A process of preparing a shuttle vector of claim 26 wherein said plasmid Pbk2s is derived from the said plasmid pBK2 of C. acetoacidophilum by
digesting said plasmid pBK2 partially with HaeII,
treating the digested plasmid with alkaline phosphatase, and
ligating the digested plasmid with origin of replication p15A obtained from E. Coli plasmid pACY184 after HaeII digestion.

31. A process of preparing a shuttle vector of claim 23 wherein said plasmid pBK2Spcs is derived from said plasmid pBK2s by
digesting said plasmid pBK2 with StuI,
treating the digested plasmid with alkaline phosphatase, and
ligating the digested plasmid with spectinomycin resistance gene cassette obtained from E. coli plasmid pDG1726 after PstI digestion and blunting with Klenow polymerase.

32. A process of preparing a shuttle vector of claim 24 wherein said plasmid pBK2Spcs is derived from said plasmid pBK2s by
digesting said plasmid pBK2 with StuI,
treating the digested plasmid with alkaline phosphatase, and
ligating the digested plasmid with spectinomycin resistance gene cassette obtained from E. coli plasmid pDG1726 after PstI digestion and blunting with Klenow polymerase.

33. A process of preparing a shuttle vector of claim 25 wherein said plasmid pBK2Spcs is derived from said plasmid pBK2s by
digesting said plasmid pBK2 with StuI,
treating the digested plasmid with alkaline phosphatase, and
ligating the digested plasmid with spectinomycin resistance gene cassette obtained from E. coli plasmid pDG1726 after PstI digestion and blunting with Klenow polymerase.

34. A process of preparing a shuttle vector of claim 26 wherein said plasmid pBK2Spcs is derived from said plasmid pBK2s by
digesting said plasmid pBK2 with StuI,
treating the digested plasmid with alkaline phosphatase, and
ligating the digested plasmid with spectinomycin resistance gene cassette obtained from E. coli plasmid pDG1726 after PstI digestion and blunting with Klenow polymerase.

35. A process as claimed in claim 23 wherein said plasmid pBKET29aS is derived from the said plasmid pET29a and pBK2Spcs by:
Digesting said plasmid pETa with DRAIII,
Blunting the digested plasmid with Klenow polymerase,
Digesting said blunted plasmid with SphI,
Gel eluting the smallest fragment containing T7 promoter cassette,
Digesting said plasmid pBK2Spcs with MluI,
Blunting the digested plasmid with Klenow polymerase,
Digesting said blunted plasmid with SphI,
Gel eluting the largest fragment, and
Ligating the digested fragments obtained from pET29a and pBK2Spcs.

36. A process as claimed in claim 24 wherein said plasmid pBKET29aS is derived from the said plasmid pET29a and pBK2Spcs by:
Digesting said plasmid pETa with DRAIII,
Blunting the digested plasmid with Klenow polymerase,
Digesting said blunted plasmid with SphI,
Gel eluting the smallest fragment containing T7 promoter cassette,
Digesting said plasmid pBK2Spcs with MluI,
Blunting the digested plasmid with Klenow polymerase,
Digesting said blunted plasmid with SphI,
Gel eluting the largest fragment, and
Ligating the digested fragments obtained from pET29a and pBK2Spcs.

37. A process as claimed in claim 25 wherein said plasmid pBKET29aS is derived from the said plasmid pET29a and pBK2Spcs by:
Digesting said plasmid pETa with DRAIII,
Blunting the digested plasmid with Klenow polymerase,
Digesting said blunted plasmid with SphI,
Gel eluting the smallest fragment containing T7 promoter cassette,
Digesting said plasmid pBK2Spcs with MluI,
Blunting the digested plasmid with Klenow polymerase,
Digesting said blunted plasmid with SphI,
Gel eluting the largest fragment, and
Ligating the digested fragments obtained from pET29a and pBK2Spcs.

38. A process as claimed in claim 26 werein said plasmid pBKET29aS is derived from the said plasmid pET29a and pBK2Spcs by:
Digesting said plasmid pETa with DRAIII,
Blunting the digested plasmid with Klenow polymerase,
Digesting said blunted plasmid with SphI,
Gel eluting the smallest fragment containing T7 promoter cassette,
Digesting the said plasmid pBK2Spcs with MluI,
Blunting the digested plasmid with Klenow polymerase,
Digesting said blunted plasmid with SphI,
Gel eluting the largest fragment, and
Ligating the digested fragments obtained from pET29a and pBK2Spcs.


SEQUENCE LISTING (4 sequences)

SEQ ID NO: 1 - DNA, 105 bp, Artificial
  Synthetic oligonucleotide
agatcgatctcgatgccgcgaaattaatacgactcactataggggaattgtcagcggataacaattcccctgtagaaataattttgtttaactttaagaaggaga

SEQ ID NO: 2 - DNA, 86 bp, Artificial
  Synthetic oligonucleotide
tatagatatgaataaacgctgcggctaaattcgacggccaggacatggacagcccagatctgggtaccctggtgccacgcggttcc

SEQ ID NO: 3 - DNA, 101 bp, Artificial
  Synthetic oligonucleotide
atgcctgatatcggatccgaattcgagctgcgtgcacaagcttgcgggcgcactcgagcaccaccaccaccaccactgagatcgggctgctaacaaagccc

SEQ ID NO: 4 - DNA, 88 bp, Artificial
  Synthetic oligonucleotide
gaaagcaagcygagtyggctgcygccaccattgtccaataattggcatgccccttcgcgcgtctacacgcgtcytgaggggttttttg